(19) United States
(12) Patent Application Publication
(10) Pub. No.: US 2011/0300227 A1
(43) Pub. Date: Dec. 8, 2011

(54) MALIGNANT NEOPLASM TREATMENT PROTOCOL

(75) Inventor: Ivan E. Danhof, Grand Prairie, TX (US)

(73) Assignee: NORTH TEXAS MEDICAL ASSOCIATES, GRAND PRAIRIE, TX (US)

(21) Appl. No.: 13/112,501

(22) Filed: May 20, 2011

Related U.S. Application Data
(60) Provisional application No. 61/347,296, filed on May 21, 2010.

(51) Int. Cl. A61K 36/185; A61K 33/00; A61K 31/385; A61K 33/34; A61K 36/48; A61K 36/28; A61K 36/31; A61K 36/42; A61K 36/23; A61K 36/45; A61K 36/889; A61K 36/55; A61K 36/752; A61K 36/736; A61K 36/73; A61K 36/21; A61K 36/8962; A61K 36/288; A61K 36/60; A61K 36/61; A61K 36/04; A61K 36/03; A61K 36/63; A61K 36/44; A61K 36/89; A61P 35/00; A61P 35/02; A61K 36/82; A61K 31/375; A61K 31/353; A61K 31/4172; A61K 31/401; A61K 31/405; A61K 36/9064; A61K 36/81; A61K 36/9068; A61K 36/537; A61K 36/53; A61K 36/9066; A61K 31/732; A61K 31/365; A61K 35/12; A61K 35/60; A61K 31/736

(52) U.S. Cl. 424/523; 514/54; 424/717; 514/440; 424/638; 424/754; 424/729; 514/474; 514/456; 514/400; 514/423; 514/419; 424/756; 424/760; 424/746; 424/745; 514/450; 424/520

(57) ABSTRACT

Methods, compositions, and strategies for the treatment of malignant neoplasms are presented herein. The treatment modalities are directed towards exploiting characteristics of cancer cells as well as correcting defective biochemical pathways and systems in the body.

### CROSS-REFERENCE TO RELATED APPLICATIONS

This patent application is a non-provisional application of U.S. Provisional Patent Application No. 61/347,296 filed on May 21, 2010 and entitled “Malignant Neoplasm Treatment Protocol” which is hereby incorporated by reference in its entirety.

### TECHNICAL FIELD OF THE INVENTION

The present invention relates in general to the field of cancer treatment, and more particularly, to a treatment modality for malignancies based on diet and exploitation of cancer cell characteristics.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with treatment modalities, compositions, and methods for treating malignant neoplasms.

U.S. Pat. No. 7,196,072 issued to Pasco et al. (2007) describes a complex, water soluble polysaccharide fraction having potent immunostimulatory activity isolated from Aloe vera. The polysaccharide fraction has an apparent molecular weight above 2 million daltons with glucose, galactose, mannose, and arabinose as its major components. The invention further describes pharmaceutical compositions containing the instant polysaccharide fraction, optionally in combination with acceptable pharmaceutical carriers and/or excipients. These pharmaceutical compositions may be used to provide immunostimulation to an individual in need of such treatment by administering to such an individual an effective amount of the composition.

U.S. Pat. No. 6,436,679 issued to Qiu and Mahiou (2002) provides a rapid and efficient method for the preparation and isolation of biologically active polysaccharides from Aloe. The Qiu patent includes the activated mixture of polysaccharides (referred to as “Immuno-10”), produced by the methods of the invention. The invention also includes the use of the polysaccharides as immunostimulating, immunomodulating, and wound healing agents. The resulting immunomodulatory complex has a higher activity and is more stable than bulk carbohydrates isolated using prior art alcohol precipitation schemes.

U.S. Pat. No. 5,296,216 issued to Turner (1994) involves a preparation adapted for prophylaxis and treatment of oral lesions. The preparation is suitable for use as an oral lavage, and comprises water, hydrogen peroxide in a premixed aqueous form in the preparation mixture, with between about 0.1% and about 0.4% sodium bicarbonate. To produce such a preparation adapted for the prophylaxis and treatment of oral lesions most preferably involves dissolving hydrogen peroxide and sodium bicarbonate in an aqueous solution to produce a premixed preparation having between about 0.1% and about 0.8% hydrogen peroxide and between about 0.1% and about 0.4% sodium bicarbonate. In more preferable embodiments of the '216 patent, the oral lavage (formulation) includes hydrogen peroxide at a concentration of about 0.4% and sodium bicarbonate at a concentration of about 0.2%. Additionally, a method for prophylaxis and treatment of oral lesions incident the use of chemotherapeutic agents is included in the present invention. This method involves the step of initially providing a premixed preparation comprising water, between about 0.1% and about 0.8% hydrogen peroxide and between about 0.1% and about 0.4% sodium bicarbonate. Oral rinsing with said preparation, particularly multiple daily oral rinsing, is shown to enhance healing of oral lesions and impedes or prevents the development of oral lesions.

U.S. Patent Publication No. 20060270625 (Vinik and Jacot, 2006) discloses a novel combination of nutraceuticals. The invention also provides novel kits comprising a novel combination of nutraceuticals. The invention also provides methods for treating symptoms or delaying the progression of neuropathy, e.g., diabetic neuropathy, ischemic neuropathy, metabolic neuropathy, neuropathy due to aging, HIV neuropathy, chemotherapeutic-induced neuropathy, para-neoplastic neuropathy, metabolic neuropathy, and the like.

WIPO Patent Application WO/2006/108429 (Rath et al. 2006) relates to the use of a composition comprising an ascorbic acid compound, a L-lysine compound, a L-proline compound, and a polyphenol compound for the preparation of a pharmaceutical composition for treating fibro- or synovial, sarcoma and prostate cancer. Moreover, the invention further relates to a method of treatment wherein said composition is administered to a subject suffering from fibro- or synovial sarcoma or prostate cancer.

### SUMMARY OF THE INVENTION

The present invention describes four treatment modalities and methods and compositions based on these modalities for treatment of cancer. The invention presents four hypotheses regarding cancer cell characteristics: (i) a defective immune surveillance system fails to detect the growth of cancer cells, (ii) cancer cells are able to survive and grow only in an acid environment, (iii) cancer cells make the mitochondria inoperable and dormant, and (iv) cancer cells require a significant increase in blood supply in order to metabolize, enlarge, and spread. The inventors further present compositions and methods for cancer treatment based on the hypothesis presented hereinabove.

In one embodiment the instant invention discloses method for treating one or more cancers in a patient comprising a combination of one or more treatment modalities selected from the group consisting of: (i) administering a radiation therapy; (ii) administering a chemotherapy; (iii) performing a surgical intervention; (iv) injecting a sterile polymannan extract formulation two to three times in a week, wherein the sterile injectable polymannan extract formulation comprises a specified quantity of very fine polymannan extract dissolved in deionized water; and one or more pharmaceutical preservatives; (v) administering an alkaline diet by itself or as a supplement along with a regular diet to create an alkaline milieu, maintain an alkaline milieu or both, wherein the alkaline diet comprises: a solution of sodium bicarbonate, one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon, and one or more optional flavoring agents, coloring agents, sweeteners, and other organoleptic compounds; (vi) administering a therapeutically effective amount of a pharmaceutical composition orally or parenterally, wherein the composition comprises: a specified quantity of dichloroacetate (DCA) in a solid or liquid dosage form, a specified quantity of alpha lipoic acid (ALA) in a solid or liquid dosage form, and one or more optional pharmaceutically acceptable excipients selected from the group consisting of preservatives, bulking agents, anti adherents, lubricants, sweeteners, and other organoleptic agents; and (vii) administering a nutritional composition comprising:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg. |

In one aspect of the method disclosed hereinabove the one or more cancers are selected from the group consisting of leukemias and lymphomas, prostate cancer, breast cancer, and colon cancer. In another aspect the polymannan extract is derived from an Aloe species selected from the group consisting of Aloe vera, Aloe arborescens, Aloe aristata, Aloe dichotoma, Aloe nyeriensis, Aloe variegate, Aloe barbadensis, and Aloe wildii. In another aspect the polymannan extract comprises aloe polysaccharides, wherein the aloe polysaccharides comprise one or more small chain, medium chain, large chain, very-large chain polysaccharides, or any combinations thereof. In related aspects the polymannan causes a 75-80% increase in one or more natural killer (NK) cells, and an increase in a level of a caspase 3 protein in the blood, wherein an increased level in caspase 3 is directly related to an increased level of apoptosis of the one or more cancer cells. In yet another aspect the administration of the alkaline diet comprises an optional step of injecting a 5% sterile solution of sodium bicarbonate to help create or maintain the alkaline milieu in the patient.

In one aspect the pharmaceutical composition prevents or slows a growth and a proliferation of one or more cancer cells, induces an apopotosis of the one or more cancer cells or both. In another aspect the nutritional composition prevents angiogenesis, a growth, a proliferation or both of the one or more cancer cells. In yet another aspect the nutritional composition is administered once or multiple times in a day before or after meals.

Another embodiment of the instant invention is related to an alkaline diet for the treatment of one or more malignancies selected from the group consisting of leukemias and lymphomas, prostate cancer, breast cancer, and colon cancer, and for the treatment of one or more immune disorders comprising: a solution of sodium bicarbonate; one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and one or more optional flavoring agents, coloring agents, sweeteners, and other organoleptic compounds.

The diet described herein is administered by itself or as a supplement along with a regular diet comprising a balanced proportion of nutritional elements selected from the group consisting of proteins, fats, carbohydrates, fiber, vitamins, and minerals. In one aspect a patient or a subject on the alkaline diet is required to have a normal to above normal fluid intake, wherein the fluids comprise water, fruit juices, nutritional drinks, milk and combinations thereof.

Yet another embodiment of the present invention relates to a method of treating a patient suffering from one or more malignancies selected from the group consisting of leukemias and lymphomas, prostate cancer, breast cancer, and colon cancer, and one or more immune disorders comprising the steps of: identifying an individual in need of treatment against the one or more malignancies; and administering an alkaline diet by itself or as a supplement along with a regular diet, wherein the alkaline diet comprises: (i) a solution of sodium bicarbonate; (ii) one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and (iii) one or more optional flavoring agents, coloring agents, sweeteners, and other organoleptic compounds. A patient undergoing the treatment described above is required to have a normal to above normal fluid intake, wherein the fluids comprise water, fruit juices, nutritional drinks, milk and combinations thereof.

An injectable solution of sodium bicarbonate for a treatment of one or more malignancies or for maintaining an alkaline milieu in a patient undergoing an alkaline diet treatment for the one or more malignancies is also disclosed in the present invention. The solution comprises: a specified quantity of sodium bicarbonate dissolved in deionized water to give a final concentration of 5%; and one or more pharmaceutical preservatives. In one aspect the injectable solution is sterile. In another aspect the one or more pharmaceutical preservatives are selected from the group consisting of parabens, benzoic acid and their salts, mercurials, quarternary ammonium salts, benzyl alcohol and other related alcohols, and phenols.

In one aspect the alkaline diet comprises: a solution of sodium bicarbonate, one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon, and one or more optional flavoring agents, coloring agents, sweeteners, and other organoleptic compounds. In another aspect the one or more malignancies selected from the group consisting of leukemias and lymphomas, prostate cancer, breast cancer, and colon cancer.

Another embodiment of the present invention provides a method of creating, maintaining an alkaline milieu or both for the treatment of one or more malignancies selected from the group consisting of leukemias and lymphomas, prostate cancer, breast cancer, and colon cancer, and one or more immune disorders in a patient comprising the steps of: identifying an individual in need of treatment against the one or more malignancies; and injecting intravenously a sterile 5% solution of sodium bicarbonate in deionized water along with one or more pharmaceutical preservatives selected from the group consisting of parabens, benzoic acid and their salts, mercurials, quarternary ammonium salts, benzyl alcohol and other related alcohols, and phenols. In one aspect the solution is injected to create an alkaline milieu in the patient for treatment of the one or more malignancies or to maintain the alkaline milieu created by administration of an alkaline diet in the patient. The alkaline diet as described hereinabove comprises: a solution of sodium bicarbonate; one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and one or more optional flavoring agents, coloring agents, sweeteners, and other organoleptic compounds.

Yet another embodiment of the instant invention describes a pharmaceutical composition for preventing or slowing a growth and a proliferation of one or more cancer cells, induction of an apopotosis of the one or more cancer cells or both in a patient comprising: a specified quantity of dichloroacetate (DCA) in a solid or liquid dosage form; a specified quantity of alpha lipoic acid (ALA) in a solid or liquid dosage form; and one or more optional pharmaceutically acceptable excipients selected from the group consisting of preservatives, bulking agents, anti adherents, lubricants, sweeteners, and other organoleptic agents. In one aspect the composition is administered orally or parenterally. In another aspect the dosage of the DCA is 10-15 mg/kg of body weight in three divided doses. In yet another aspect the dosage of the ALA is 100 mg/kg of body weight three times a day administered after meals. The ALA is administered separately by itself or is administered in a composition combined with the DCA.

In one embodiment the instant invention is a method for treating one or more cancers by preventing or slowing a growth and a proliferation of the one or more cancer cells, induction of an apopotosis of the one or more cancer cells or both in a patient comprising the steps of: identifying an individual in need of treatment against the one or more cancers; and administering a therapeutically effective amount of a pharmaceutical composition orally or parenterally, wherein the composition comprises: a specified quantity of dichloroacetate (DCA) in a solid or liquid dosage form; a specified quantity of alpha lipoic acid (ALA) in a solid or liquid dosage form; and one or more optional pharmaceutically acceptable excipients selected from the group consisting of preservatives, bulking agents, anti adherents, lubricants, sweeteners, and other organoleptic agents. In one aspect the dosage of the DCA is 10-15 mg/kg of body weight in three divided doses and the dosage of the ALA is 100 mg/kg of body weight three times a day administered after meals. In another aspect the ALA is administered separately by itself or is administered in a composition combined with the DCA. In yet another aspect the one or more cancers comprise leukemias and lymphomas, prostate cancer, breast cancer, and colon cancer.

Another embodiment of the present invention describes a nutrient composition for preventing angiogenesis, a growth, a proliferation or both of one or more cancer cells comprising: Vitamin C, Vitamin C salts, modifications and derivatives thereof; one or more amino acids selected from the group consisting of alanine, arginine, asparagine, aspartic acid, cysteine, glycine, glutamine, glutamic acid, histidine, isoleucine, leucine, lysine, methionine, phenylalanine, proline, serine, tryptophan, threonine, tyrosine, and valine; one or more herbal, plant, and animal products comprising tea extracts, catechins, polyphenols, epigaliocatechingallate (EGCG), cardamom, cayenne pepper, ginger, sage, thyme, turmeric, citrus pectin, artemisinin, graviola, carnivora, fish oil (epa+dha), genestein (soybeans), garlic; one or more mineral supplements; one or more optional additives selected from the group consisting of coloring agents, flavoring agents, sweeteners, preservatives, and other organoleptic agents.

Yet another embodiment discloses a nutrient composition for preventing angiogenesis, a growth, a proliferation or both of one or more cancer cells comprising:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg. |

In one aspect the one or more cancers comprise leukemias and lymphomas, prostate cancer, breast cancer, and colon cancer. In another aspect the composition is administered once or multiple times in a day. In another aspect the composition is administered before or after meals by itself or as part of a combination cancer therapy comprising a radiation therapy, a chemotherapy, a surgical procedure, an injection of a polymannan extract, an alkaline diet therapy, administration of dichloroacetate (DCA), and combinations and modifications thereof.

Another embodiment of the present invention provides a method for treating one or more cancers by preventing angiogenesis, a growth, a proliferation or both of one or more cancer cells in a patient comprising the steps of: identifying the patient in need of treatment against the one or more cancers, wherein the one or more cancers comprise leukemias and lymphomas, prostate cancer, breast cancer, and colon cancer and administering a nutritional composition for the prevention of the angiogenesis, the growth, the proliferation or both of one or more cancer cells in the patient. The composition comprises:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg. |

In one aspect the composition is administered once or multiple times in a day. In another aspect the composition is administered before or after meals. In yet another aspect the composition is administered by itself or as part of a combination cancer therapy comprising a radiation therapy, a chemotherapy, a surgical procedure, an injection of a polymannan extract, an alkaline diet therapy, administration of dichloroacetate (DCA), and combinations and modifications thereof.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “neoplasm” refers to any new and abnormal growth of tissue. Thus, a neoplasm can be a premalignant neoplasm or a malignant neoplasm. The term “malignant” used herein refers to properties that cancer cells exhibit, such as angiogenesis-inducing ability, hematogenous metastasis, lymphogenous metastasis, dissemination, retention of cancerous ascites or pleural effusion, cancerous cachexia and shortening of survival time of the host based thereon. The term “cancer” includes lymphomas, carcinomas and sarcomas, and other neoplastic conditions, as these terms are commonly used in the art (See, e.g. The Merck Manual, 16th Ed., supra).

The term “anaerobic” is used herein to mean substantially free of oxygen. The term “glycolysis” refers to the biochemical degradation of the body's energy reserves glycogen or starch in the human or animal body in relevant reference works (see, for example, Rompps Chemie Lexikon, 10th Edition, p. 1579). The term “mitochondria” as used herein a refers to the membrane-enclosed organelle found in most eukaryotic cells ranging from 0.5 to 10 micrometers (μm) in diameter. Mitochondria generate most of the cell's supply of adenosine triphosphate (ATP), used as a source of chemical energy. In addition to supplying cellular energy, mitochondria are involved in a range of other processes, such as signaling, cellular differentiation, cell death, as well as the control of the cell cycle and cell growth.

The term “vascular endothelial growth factor” and the abbreviation “VEGF” (without modifier) are used herein in a generic sense, to describe any of a family of growth factor polypeptides including but not limited to Vascular Endothelial Growth Factor-A (VEGF-A), Vascular Endothelial Growth Factor-B (VEGF-B), Vascular Endothelial Growth Factor-C (VEGF-C), Vascular Endothelial Growth Factor-D (VEGF-D), Platelet Derived Growth Factor-A (PDGF-A), Platelet Derived Growth Factor-B (PDGF-B), Placenta Growth Factor (PlGF), and virally encoded VEGF-like molecules.

The term “Aloe” refers to the genus of South African plants of the Liliaceae family of which the Aloe barbadensis plant is a species.

The terms “administration of” or “administering a” compound refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

As used herein, the term “intravenous” refers to a mode of administration of a substance such as a drug or a nutrient solution, within or into a vein. As used herein, the term “treatment” or “treating” means any administration of a compound of the present invention and includes (1) inhibiting the disease in an animal that is experiencing or displaying the pathology or symptomatology of the disease (i.e., arresting further development of the pathology and/or symptomatology), or (2) ameliorating the disease in an animal that is experiencing or displaying the pathology or symptomatology of the disease (i.e., reversing the pathology and/or symptomatology).

The term “pharmaceutically acceptable” as used herein refers to the carrier, diluent or excipient that must be compatible with the other ingredients of the formulation and not deleterious to the recipient thereof.

As used herein, the term “treatment” or “treating” refers to administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The present invention describes new compositions and regimens for the treatment of malignant neoplasms. The treatment strategies are based on four hypotheses presented by the present inventors regarding characteristics of cancer cells and inherent metabolic deficiencies that permit the enlargement and spread of cancer cells. The hypotheses presented are presented herein below:

I. Defective immune surveillance function which fails to detect the presence of cancerous cells and fails to destroy them.

II. Cancer cells are able to survive and grow only in an acidic environment.

III. Cancer cells utilize anaerobic (no oxygen) glycolysis (breakdown of sugars) as their method of producing energy for sustained growth and metastatic spread. To achieve this state, the cancer cells produce substances which make the mitochondria (oxygen-utilizing metabolic organelles) inoperable and dormant.

IV. Cancer cells require a significant increase in blood supply in order to metabolize, enlarge, and spread. The tumors produce materials which stimulate vascular-endothelial-growth factor (VEGF) which is largely responsible for the formation of new blood vessels (angiogenesis).

To address the defective immune surveillance system to cure the cancers the inventors suggest Polymannan Extract (PME) i.v. injections. PME comprising an array of glucomannan polysaccharides derived from Aloe barbadensis Miller according to a previously patented method (U.S. Pat. No. 6,083,508 (2000)) by the present inventors. The polysaccharides range in molecular size from about 100,000 Daltons to 10,000,000 Daltons. Some calcium and magnesium salts are also present which stabilize the product.¹⁻³

TABLE 1
Compositions and molecular weights of aloe polysaccharides
having Mol. Wt's of 100,000 or greater.

| Parameter | Molecular Size Range | Sugar Moieties |
|---|---|---|
| Aloeride | 2.0 × 10⁶-10.0 × 10⁶ | Predominantly mannose and |
| | | glucose; traces of arabinose |
| | | and galactose |
| Acemannan | 9.0 × 10⁵-1.5 × 10⁶ | Mannose, glucose |
| Manapol | 5.0 × 10⁵-9.0 × 10⁵ | Mannose, glucose |
| Aloemannan | 1.0 × 10⁵-5.0 × 10⁵ | Mannose, glucose |

The longer the polysaccharide chain and the greater the content of mannose, the greater the immunomodulatory activity of the preparations. The product is available as a sterile solution, 10 mg/mL, in 10 mL multidose vials.

Mechanism of Action of PME: PME, via its mannose moieties, binds to mannose receptors on the surface of the monocytes/macrophages, which thereupon induces the release of an array of cytocommunicators, including TNF-α, IL-β, INF-γ, IL-2, and IL-6.

Delineated Responses of Stimulated Cytocommunicators: a. Increased cell counts of monocytes/macrophages, b. Increased cytokine production, c. Increased expression of cellular surface molecules, d. Increased numbers of Natural Killer (NK) cells, e. Increased phagocytosis activity, f. Increased anti-viral activity, and g. Increased potent anti-tumor activity.

Treatment Schedule: The patient initially undergoes daily intravenous administrations to determine the dosage to induce the “Physiological (Fever Response) reaction”. When PME combines with receptors on the monocytes/macrophages and Tumor Necrosis Factor—Alpha and Interleukin 1-Beta are released, this simulates what happens when a bacterial or viral organism invades the body and alerts the immune system response. The “Fever Response” is due to the resetting of the hypothalamic temperature-regulating centers so the body tries to retrain more heat and/or decrease heat loss from the body to elevate the body temperature, which is one of nature's most common responses, as increased body temperature may create an adverse environment for the invader. This is associated with feelings of chilliness, coldness, shivers, shakes, and occasionally even rigors depending upon the degree of response in a given patient.

On Day 1, the patient receives 0.1 mL (equivalent to 1 mg PME) intravenously; on each succeeding day the dosage is increased by 0.1 mL until a dosage of 1.0-1.5 mL is reached. If the patient is relatively robust, the dosage schedule selected may be 0.1 mL, 0.3 mL, 0.5 mL, 0.75 mL, 1.0 mL, 1.25 mL and 1.5 mL on succeeding days. Once the Fever Response is observed, often that dosage is maintained on the two, three, or more days per week schedule. For mild disease states 2-3 injections per week are used; for moderate severity states, 3-4 injections per week are recommended; with severe, advanced cases, the injections may be given 5-6 times per week.

As there are constant variables going on in the patient, oftentimes the patient may react on one occasion and not on other occasions; the absence of a reaction does not mean the product is not working; it just means that the majority of binding sites available are occupied. Factors which influence the binding reactions include the white blood cell count, the differential (types of white cells present) count, number of mannose binding sites on the cells, etc. Once binding occurs, the influence on the white cell may last 12 or more hours.

Excess PME binds to a circulating Mannose-Binding Protein (MBP) which can provide an almost constant binding to the white cells, owing to the fact that the affinity of the PME to bind to the cells is far greater than the affinity of binding to this carrier protein.

The present inventors hypothesize that cancer cells are able to survive and grow only in an acidic environment. The inventors propose a treatment regimen wherein the pH (acid-alkali reaction) of arterial blood is kept nearly constant at 7.35, a mildly alkaline state by a carefully regulated sequence of physiological processes, but the pH in the interstitial fluid spaces, where the cells live, can be quite acidic. The tumors, themselves, produce acids derived from their metabolism to maintain their acid environment.

All foods which are ingested and metabolized leave either acidic or alkaline residues—the so-called acid ash or alkaline ash residue. The inventors aim to provide the maximum of alkaline residues, the alkaline ash diet is which is recommended to all cancer patients. In addition the inventors recommend taking about ½ tsp of NaHCO₃ solution with each meal.

Alkaline ash forming foods include alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon.

Note that the meats, one of the major protein sources, are not on the list. It is permissible, however, for the patient to have one 3-ounce portion per day of beef, chicken, eggs, fish, pork, or turkey. One of the most important items is for the patient to drink lots of water, permitted fruit juices, etc.

Intravenous Sodium Bicarbonate Solution: Evidence is accruing that cancers are etiologically related to chronic fungal infections, e.g., Candida albicans. These etiological (causal) elements are killed by the administration of sterile, pyrogen-free sodium bicarbonate solution. The dose is 300-500 mL (depending upon bodyweight) of a 5% sodium bicarbonate solution given 3-5 times per week depending upon the severity of the problem. This treatment also assists in the maintenance of the alkaline milieu.

The inventors also hypothesize that the cancer cells make the normal mitochondrial cells inoperable and dormant for the growth and proliferation. Cancer cells, as well as all other abnormal cells in the body, are programmed for apoptosis (cell death). If one changes the metabolism from glycolysis to aerobic mitochondrial metabolism, the pre-programmed self destruction of the cell is initiated.

To counter this the inventors suggest reawakening and restarting the mitochondrial aerobic metabolism by the administration of dichloroacetate (DCA). DCA was widely used in the late 30's and 40's as an oral anti-inflammatory medication to treat arthritis. It was replaced by newer anti-inflammatory agents and thus has not been used for years. An interesting corollary is that this agent was associated with possibly fewer undesirable side-effects than the currently widely used NSAIDS (Non-steroidal-anti-inflammatory-drugs). In large doses, however, DCA has been associated with peripheral neuropathy (numbness and tingling in hands and feet), but this can be prevented or moderated with lower dosages. The usual dosage of DCA is 10-15 mg/kg daily in divided (three) doses. To prevent neuropathic side effects, alpha lipoic acid (ALA) should be used as a supplement. The usual dosage of ALA is 100 mg three times daily with meals. If the dosage of DCA is 10 mg/kg or less, the incidence of peripheral neuropathy is significantly reduced.

According to the present inventors, cancer cells require a significant increase in blood supply in order to metabolize, enlarge, and spread. An interesting aberration is that the primary (mother) tumor often produces ANTI-angiogenic materials so that distant metastases are suppressed in their growth because they cannot develop the additional blood supply. This means that the usual MRI or PET scans see the mother tumor, but the very small, suppressed distant metastases do not appear on the scans, but may become a plethora of tumor sites once the major tumor is excised (debulking surgical procedure). In the not too distant past, this phenomenon (i.e. the removal of the primary tumor associated with the appearance of distant metastases on scans) was viewed as a worsening of the patient's condition. It is far easier to treat the small distant metastases with our treatment protocol as they are so much smaller, and the local tumor conditions are not overwhelming the immune system mechanisms.

Treatment: To prevent the growth of the requisite increase in blood vessels that the tumors require for growth and spread, it is necessary to administer anti-angiogenesis agents. A number of these agents have been described including the following: Artimesinin, Iodine, Tetrathiomolybdate (TTM), Carnivora (a preparation using, among other materials, a derivative of Venus Flytrap) which is claimed to be a potent immune stimulator as well as an anti-angiogenesis agent, Graviola (Annona muricata) which contains acetogenins which are potent inhibitors of enzyme processes found only in cancer cells but have to toxicity for normal healthy cells), Thalidomide.

However, the inventors describe a novel nutrient mixture, which is very effective in controlling VEGF and other angiogenic agents (see Table 2).⁴

TABLE 2
Anti-angiogenesis nutrient mixture.

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg |

The Vitamin C in the novel formulation of the instant invention inhibits all division and growth of cancer cells through the production of hydrogen peroxide.⁵ The Green tea extract controls angiogenesis and metastases.⁶ The N-acetyl cysteine inhibits MMP 9 activity, blocks invasive actions of tumor cells, and inhibits endothelial invasion.⁷⁻⁹ Selenium decreases MMP expression in tumor cells and decreases migration of endothelial cells. ¹⁰⁻¹¹Arginine is a precursor of nitric oxide (NO) which induces apoptosis.¹²

There are a host of dietary and herbal supplements which are suggested as being anti-tumor, including such entities as various mushrooms, cardamom, cayenne pepper, ginger, sage, thyme, turmeric, citrus pectin, etc. which may be used on an individual basis.

Additional anti-angiogenetic materials: artemisinin, graviola, carnivora, fish oil (epa+dha), genestein (soybeans), garlic, epigaliocatechingallate (EGCG) (green tea).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,196,072: High Molecular Weight Polysaccharide Fraction from Aloe Vera with Immunostimulatory Activity.
U.S. Pat. No. 6,436,679: Process for the Preparation of Immunodulatory Polysaccharides from Aloe.
U.S. Pat. No. 5,296,216: Prevention and Treatment of Oral Lesions.
U.S. Patent Publication No. 20060270625: Nutraceuticals for the Treatment of Neuropathy.
WIPO Patent Application WO/2006/108429: Nutrient Composition for treating Sarcoma and Prostate Cancer.
¹ Leung M A, Liu C, Zhu L F, Ho Y Z, Yu B, Fung K P: Chemical And Biological Characterization Of A Polysaccharide Biological Response Modifier From Aloe Vera L. Glycobiology 14(6):501-510, 2004.
²Pugh N., Ross S A, ElSohly M A, and Pasco, D S (2004). Characterization of Aloeride, a new high-molecular weight polysaccharide from Aloe vera with potent immunostimulating activity. J. Agr. Food Chem., 49(2), 1030-1034.
³ Im S A, Oh S T, Song S, Kim M R, Kim D S, Woo S S, Jo T H, Park Y I, Lee C K (2005). Identification of optimal molecular size of modified Aloe polysaccharide with maximum immunostimulant activity. International Immunopharmacology Journal 5(2):271-279.
⁴ Roomi M W, Ivanov V, Kalinovsky T et al. A novel nutrient mixture containing ascorbic acid, lysine, proline, and green tea extract inhibits critical parameters in angiogenesis. In Losso I N, Shahidi F, Bagchi D: Antit-angiogenic functional and medicinal foods. Boca Raton, Fla., CRC Press, 2007, pp. 561-580.
⁵ Prostate 32:188-195, 1997.
⁶ Hare F: Green Tea: Health Benefits and Applications. Marcel Dekker, New York and Basel, 2001).
⁷ Anticancer research 21:213-219, 2001.
⁸International Journal of Biological Markers, 14: 268-271, 1999
⁹ Journal of Biological Chemistry 276:20085-20092, 2001.
¹⁰ Journal of Biological Chemistry 276: 20085-20092, 2001.
¹¹ FASB 16:2-14, 2002, International Journal of Biological Markers, 14:268-271, 1999.
¹² Annal Review of Medicine: 48:489-509, 1997.

What is claimed is:

1. A method for treating one or more cancers in a patient comprising a combination of one or more treatment modalities selected from the group consisting of:
administering a radiation therapy;
administering a chemotherapy;
performing a surgical intervention;
injecting a sterile polymannan extract formulation two to three times a week, wherein the sterile injectable polymannan extract formulation comprises a specified quantity of a very fine polymannan extract dissolved in deionized water and one or more pharmaceutical preservatives;
administering an alkaline diet by itself or as a supplement along with a regular diet to create an alkaline milieu, maintain an alkaline milieu, or both, wherein the alkaline diet comprises: a solution of sodium bicarbonate, one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon, and one or more optional flavoring agents, coloring agents, sweeteners, and other organoleptic compounds;
administering a therapeutically effective amount of a pharmaceutical composition orally or parenterally, wherein the composition comprises: a specified quantity of dichloroacetate (DCA) in a solid or liquid dosage form, a specified quantity of alpha lipoic acid (ALA) in a solid or liquid dosage form, and one or more optional pharmaceutically acceptable excipients selected from the group consisting of preservatives, bulking agents, anti adherents, lubricants, sweeteners, and other organoleptic agents; and
administering a nutritional composition comprising:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg. |

2. The method of claim 1, wherein the one or more cancers are selected from the group consisting of leukemias and lymphomas, prostate cancer, breast cancer, and colon cancer.

3. The method of claim 1, wherein the polymannan extract is derived from an Aloe species selected from the group consisting of Aloe vera, Aloe arborescens, Aloe aristata, Aloe dichotoma, Aloe nyeriensis, Aloe variegate, Aloe barbadensis, and Aloe wildii.

4. The method of claim 1, wherein the polymannan extract comprises aloe polysaccharides, wherein the aloe polysaccharides comprise one or more small chain, medium chain, large chain, very-large chain polysaccharides, or any combinations thereof.

5. The method of claim 1, wherein the polymannan extract causes a 75-80% increase in one or more natural killer (NK) cells.

6. The method of claim 1, wherein the polymannan extract increases a level of a caspase 3 protein in the blood, wherein an increased level in caspase 3 is directly related to an increased level of apoptosis of the one or more cancer cells.

7. The method of claim 1, wherein the administration of the alkaline diet comprises an optional step of injecting a 5% sterile solution of sodium bicarbonate to help create or maintain the alkaline milieu in the patient.

8. The method of claim 1, wherein the pharmaceutical composition prevents or slows a growth and a proliferation of one or more cancer cells, induces an apopotosis of the one or more cancer cells, or both.

9. The method of claim 1, wherein the nutritional composition prevents angiogenesis, a growth, a proliferation or both of the one or more cancer cells.

10. The method of claim 1, wherein the nutritional composition is administered once or multiple times in a day before or after meals.

11. An alkaline diet for the treatment of one or more malignancies selected from the group consisting of leukemias and lymphomas, prostate cancer, breast cancer, and colon cancer, and for the treatment of one or more immune disorders comprising:
a solution of sodium bicarbonate;
one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and
one or more optional flavoring agents, coloring agents, sweeteners, and other organoleptic compounds.

12. The diet of claim 11, wherein the diet is administered by itself or as a supplement along with a regular diet comprising a balanced proportion of nutritional elements selected from the group consisting of proteins, fats, carbohydrates, fiber, vitamins, and minerals.

13. The diet of claim 11, wherein a patient or a subject on the alkaline diet is required to have a normal to above normal fluid intake, wherein the fluids comprise water, fruit juices, nutritional drinks, milk, and combinations thereof.

14. A method of treating a patient suffering from one or more malignancies selected from the group consisting of leukemias and lymphomas, prostate cancer, breast cancer, and colon cancer, one or more immune disorders, or both comprising the steps of:
identifying an individual in need of treatment against the one or more malignancies, the one or more immune disorders, or both; and
administering an alkaline diet by itself or as a supplement along with a regular diet, wherein the alkaline diet comprises:
a solution of sodium bicarbonate;
one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and
one or more optional flavoring agents, coloring agents, sweeteners, and other organoleptic compounds.

15. The method of claim 14, wherein the patient is required to have a normal to above normal fluid intake, wherein the fluids comprise water, fruit juices, nutritional drinks, milk and combinations thereof.

16. An injectable solution of sodium bicarbonate for a treatment of one or more malignancies or for maintaining an alkaline milieu in a patient undergoing an alkaline diet treatment for the one or more malignancies comprising:
a specified quantity of sodium bicarbonate dissolved in deionized water to give a final concentration of 5%; and
one or more pharmaceutically acceptable preservatives.

17. The solution of claim 16, wherein the injectable solution is sterile.

18. The solution of claim 16, wherein the one or more pharmaceutically acceptable preservatives are selected from the group consisting of parabens, benzoic acid and their salts, mercurials, quarternary ammonium salts, benzyl alcohol and other related alcohols, and phenols.

19. The solution of claim 16, wherein the alkaline diet comprises
a solution of sodium bicarbonate;
one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and
one or more optional flavoring agents, coloring agents, sweeteners, and other organoleptic compounds.

20. The solution of claim 16, wherein the one or more malignancies selected from the group consisting of leukemias and lymphomas, prostate cancer, breast cancer, and colon cancer.

21. A method of creating, maintaining an alkaline milieu, or both for the treatment of one or more malignancies selected from the group consisting of leukemias and lymphomas, prostate cancer, breast cancer, and colon cancer, and one or more immune disorders in a patient comprising the steps of:
identifying an individual in need of treatment against the one or more malignancies; and
injecting intravenously a sterile 5% solution of sodium bicarbonate in deionized water along with one or more pharmaceutical preservatives selected from the group consisting of parabens, benzoic acid and their salts, mercurials, quarternary ammonium salts, benzyl alcohol and other related alcohols, and phenols.

22. The method of claim 21, wherein the solution is injected to create an alkaline milieu in the patient for treatment of the one or more malignancies or to maintain the alkaline milieu created by administration of an alkaline diet in the patient.

23. The method of claim 22, wherein the alkaline diet comprises:
a solution of sodium bicarbonate;
one or more alkaline ash forming foods selected from the group consisting of alfalfa sprouts, artichokes, broccoli, cantaloupe, celeriac, cranberries, dates, flaxseed, huckleberries, lemons, almonds, apples (apple cider) apricots, bananas, beets, blackberries, blueberries, brussel sprouts, burdock cabbage, carob carrots, cauliflower, celery, chard, cherries, chives, coconut, cucumbers, currants, dandelion greens, dill, dock, endive, figs (dried), garlic, grapefruit, green beans, guava, irish moss, kelp, kohlrabi leeks, lettuce, lima beans, limes, loganberries, loquats, molasses, nectarines, oranges, pears, radishes, rutabagas, squash, vegetable oils, mango, melons, millet mint, mulberries, muskmelon, mustard greens, okra, olives, olive oil, onions, papaya, parsley, parsnips, peaches, persimmons, pineapple, plums, pumpkin, raisins, raspberries, rhubarb, romaine, sea grass, sorrell, soybeans, spinach, strawberries, swisschard, tangerines, turnips, water chestnuts, watercress, and watermelon; and
one or more optional flavoring agents, coloring agents, sweeteners, and other organoleptic compounds.

24. A pharmaceutical composition for preventing or slowing a growth and a proliferation of one or more cancer cells, induction of an apopotosis of the one or more cancer cells or both in a patient comprising:
a specified quantity of dichloroacetate (DCA) in a solid or liquid dosage form;
a specified quantity of alpha lipoic acid (ALA) in a solid or liquid dosage form; and
one or more optional pharmaceutically acceptable excipients selected from the group consisting of preservatives, bulking agents, anti adherents, lubricants, sweeteners, and other organoleptic agents.

25. The composition of claim 24, wherein the composition is administered orally or parenterally.

26. The composition of claim 24, wherein a dosage of the DCA is 10-15 mg/kg of body weight in three divided doses.

27. The composition of claim 24, wherein the dosage of the ALA is 100 mg/kg of body weight administered three times a day after meals.

28. The composition of claim 24, wherein the ALA is administered separately by itself or is administered in a composition combined with the DCA.

29. A method for treating one or more cancers by preventing or slowing a growth and a proliferation of the one or more cancer cells, induction of an apopotosis of the one or more cancer cells, or both in a patient comprising the steps of:
identifying an individual in need of treatment against the one or more cancers; and
administering a therapeutically effective amount of a pharmaceutical composition orally or parenterally, wherein the composition comprises:
a specified quantity of dichloroacetate (DCA) in a solid or liquid dosage form;
a specified quantity of alpha lipoic acid (ALA) in a solid or liquid dosage form; and
one or more optional pharmaceutically acceptable excipients selected from the group consisting of preservatives, bulking agents, anti adherents, lubricants, sweeteners, and other organoleptic agents.

30. The method of claim 29, wherein a dosage of the DCA is 10-15 mg/kg of body weight in three divided doses.

31. The method of claim 29, wherein the dosage of the ALA is 100 mg/kg of body weight administered three times a day after meals.

32. The method of claim 29, wherein the ALA is administered separately by itself or is administered in a composition combined with the DCA.

33. The method of claim 29, wherein the one or more cancers comprise leukemias and lymphomas, prostate cancer, breast cancer, and colon cancer.

34. A nutrient composition for preventing angiogenesis, a growth, a proliferation, or both of one or more cancer cells comprising:
Vitamin C, Vitamin C salts, modifications and derivatives thereof;
one or more amino acids selected from the group consisting of alanine, arginine, asparagine, aspartic acid, cysteine, glycine, glutamine, glutamic acid, histidine, isoleucine, leucine, lysine, methionine, phenylalanine, proline, serine, tryptophan, threonine, tyrosine, and valine;
one or more herbal, plant, and animal products comprising tea extracts, catechins, polyphenols, epigaliocatechingallate (EGCG), cardamom, cayenne pepper, ginger, sage, thyme, turmeric, citrus pectin, artemisinin, graviola, carnivora, fish oil (epa+dha), genestein (soybeans), garlic;
one or more mineral supplements; and
one or more optional additives selected from the group consisting of coloring agents, flavoring agents, sweeteners, preservatives, and other organoleptic agents.

35. A nutrient composition for preventing angiogenesis, a growth, a proliferation or both of one or more cancer cells comprising:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg. |

36. The composition of claim 35, wherein the one or more cancers comprise leukemias and lymphomas, prostate cancer, breast cancer, and colon cancer.

37. The composition of claim 35, wherein the composition is administered once or multiple times in a day.

38. The composition of claim 35, wherein the composition is administered before or after meals.

39. The composition of claim 35, wherein the composition is administered by itself or as part of a combination cancer therapy.

40. The composition of claim 39, wherein the cancer therapy comprises a radiation therapy, a chemotherapy, a surgical procedure, an injection of a polymannan extract, an alkaline diet therapy, administration of dichloroacetate (DCA), and combinations and modifications thereof.

41. A method for treating one or more cancers by preventing angiogenesis, a growth, a proliferation, or both of one or more cancer cells in a patient comprising the steps of:
identifying the patient in need of treatment against the one or more cancers, wherein the one or more cancers comprise leukemias and lymphomas, prostate cancer, breast cancer, and colon cancer; and
administering a nutritional composition comprising:

| Vitamin C (Ascorbic acid, magnesium | 700 | mg |
|---|---|---|
| ascorbate, calcium ascorbate, and palmitic ascorbate) | | |
| L-Lysine | 1,000 | mg |
| L-Proline | 750 | mg |
| L-Arginine | 500 | mg |
| N-Acetyl Cysteine | 200 | mg |
| Green tea Extract | 1000 | mg |
| Total polyphenols | 80% | |
| Catechins | 60% | |
| EGCG | 35% | |
| Caffeine | 1% | |
| Minerals | | |
| Selenium | 30 | mcg |
| Copper | 2 | mg |
| Manganese | 1 | mg |

for the prevention of the angiogenesis, the growth, the proliferation or both of one or more cancer cells in the patient.

42. The method of claim 41, wherein the composition is administered once or multiple times in a day.

43. The method of claim 41, wherein the composition is administered before or after meals.

44. The method of claim 41, wherein the composition is administered by itself or as part of a combination cancer therapy.

45. The method of claim 44, wherein the cancer therapy comprises a radiation therapy, a chemotherapy, a surgical procedure, an injection of a polymannan extract, an alkaline diet therapy, administration of dichloroacetate (DCA), and combinations and modifications thereof.
